(19) United States
(12) Patent Application Publication
(10) Pub. No.: US 2024/0164648 A1
(43) Pub. Date: May 23, 2024

(54) SAFETY METHODS FOR DEVICES CONFIGURED TO EMIT HIGH-INTENSITY LIGHT

(71) Applicant: QUALCOMM Incorporated, San Diego, CA (US)

(72) Inventors: Justin TSE, Poway, CA (US); Htet NAING, San Diego, CA (US); Sherman Sebastian ANTAO, San Diego, CA (US); Nicholas BUCHAN, San Jose, CA (US); Ye ZHAN, Buffalo, NY (US)

(21) Appl. No.: 18/057,484

(22) Filed: Nov. 21, 2022

(51) Int. Cl. A61B 5/00

(52) U.S. Cl. CPC A61B 5/0095; A61B 5/024; A61B 5/6826

(57) ABSTRACT

Some disclosed devices include a light source system, a target detection system; and a control system configured to communicate with the light source system and the target detection system. The control system may be further configured to receive target detection data from the target detection system, to estimate the presence or absence of a biological target based, at least in part, on the target detection data and to enable or disable the light source system based, at least in part, on an estimation of the presence or absence of the biological target. For example, if the biological target is detected, the control system may enable the light source system.

### TECHNICAL FIELD

This disclosure relates generally to methods, devices and systems for protecting people from undesirable exposure to high-intensity light, such as protecting people's eyes from laser light.

### DESCRIPTION OF RELATED TECHNOLOGY

A variety of different sensing technologies and algorithms are being implemented in devices for various biometric and biomedical applications, including health and wellness monitoring. Some such devices use high-intensity light, such as laser light. Improved methods, devices and systems for protecting people from undesirable exposure to such high-intensity light, such as for protecting people's eyes from laser light, would be desirable.

### SUMMARY

The systems, methods and devices of this disclosure each have several aspects, no single one of which is solely responsible for the desirable attributes disclosed herein.

One innovative aspect of the subject matter described in this disclosure can be implemented in an apparatus. The apparatus may include a light source system, a target detection system and a control system configured to communicate with the light source system and the target detection system. In some implementations, a mobile device (such as a wearable device, a cellular telephone, etc.) may be, or may include, at least part of the apparatus.

The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system may be configured to receive target detection data from the target detection system. The control system may be configured to estimate a presence or absence of a biological target based, at least in part, on the target detection data. The control system may be configured to enable or disable the light source system based, at least in part, on the estimation of the presence or absence of the biological target.

In some examples, the light source system may include one or more lasers or laser diodes. According to some examples, the light source system may include one or more light-emitting diodes.

According to some examples, the control system may be further configured to control the light source system to emit one or more light pulses towards the biological target and to perform one or more types of bio-sensing functionality, biometric functionality, or combinations thereof, based on one or more responses of the biological target to the one or more light pulses. In some such examples, the apparatus may include an ultrasonic receiver system and the control system may be further configured to receive ultrasonic receiver signals from the ultrasonic receiver system corresponding to ultrasound caused by the one or more responses of the biological target to the one or more light pulses. In some such examples, the control system may be further configured to provide photoacoustic imaging functionality, photoacoustic-based blood pressure estimation functionality, a photoacoustic-based authentication process or combinations thereof, based at least in part on the ultrasonic receiver signals.

In some examples, the target detection system may include a touch sensor system, a force sensor system, one or more mechanical switches, one or more electrical switches, one or more magnetic switches, one or more magnets configured for electrical continuity, an optical sensor system, one or more cameras, or combinations thereof. According to some examples in which the target detection system includes the optical sensor system, the control system may be further configured to control an intensity of light emitted by the light source system based, at least in part, on optical sensor data from the optical sensor system.

According to some examples, the target detection system may include a liveness detection system. In some such examples, the liveness detection system may include a cardiac pulse detection system. In some such examples, the cardiac pulse detection system may include a camera system, an ultrasonic pulse detection system, an optical pulse detection system, a photoacoustic pulse detection system, a photoplethysmography system, a microphone system, a ballistocardiogram sensor system, or combinations thereof.

In some examples, the target detection system may be configured to produce a first instance of target detection data at a first time and to produce a second instance of target detection data at a second time. In some such examples, a time interval between the first time and the second time may be a target detection latency period. According to some such examples, the target detection latency period may be less than a pulse repetition frequency of the light source system.

Other innovative aspects of the subject matter described in this disclosure can be implemented in a method. The method may involve receiving, by a control system, target detection data from a target detection system. The method may involve estimating, by the control system, a presence or absence of a biological target based, at least in part, on the target detection data. The method may involve enabling or disabling the light source system, by the control system and based, at least in part, on the estimation of the presence or absence of the biological target.

In some examples, the method may involve controlling, by the control system, the light source system to emit one or more light pulses towards the biological target and performing one or more types of bio-sensing functionality, biometric functionality, or combinations thereof, based on one or more responses of the biological target to the one or more light pulses. In some examples, the method may involve receiving, by the control system, ultrasonic receiver signals from an ultrasonic receiver system corresponding to ultrasound caused by the one or more responses of the biological target to the one or more light pulses. In some examples, the method may involve providing, by the control system, photoacoustic imaging functionality, photoacoustic-based blood pressure estimation functionality, a photoacoustic-based authentication process or combinations thereof, based at least in part on the ultrasonic receiver signals.

According to some examples, the method may involve producing, by the target detection system, a first instance of target detection data at a first time and a second instance of target detection data at a second time. In some such examples, a time interval between the first time and the second time may be a target detection latency period. According to some examples, the target detection latency period may be less than a pulse repetition frequency of the light source system.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon. The software may include instructions for controlling one or more devices to perform one or more disclosed methods.

According to some such examples, the method may involve receiving, by a control system, target detection data from a target detection system. The method may involve estimating, by the control system, a presence or absence of a biological target based, at least in part, on the target detection data. The method may involve enabling or disabling the light source system, by the control system and based, at least in part, on the estimation of the presence or absence of the biological target.

In some examples, the method may involve controlling, by the control system, the light source system to emit one or more light pulses towards the biological target and performing one or more types of bio-sensing functionality, biometric functionality, or combinations thereof, based on one or more responses of the biological target to the one or more light pulses. In some examples, the method may involve receiving, by the control system, ultrasonic receiver signals from an ultrasonic receiver system corresponding to ultrasound caused by the one or more responses of the biological target to the one or more light pulses. In some examples, the method may involve providing, by the control system, photoacoustic imaging functionality, photoacoustic-based blood pressure estimation functionality, a photoacoustic-based authentication process or combinations thereof, based at least in part on the ultrasonic receiver signals.

According to some examples, the method may involve producing, by the target detection system, a first instance of target detection data at a first time and a second instance of target detection data at a second time. In some such examples, a time interval between the first time and the second time may be a target detection latency period. According to some examples, the target detection latency period may be less than a pulse repetition frequency of the light source system.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1A shows an example of a blood pressure monitoring device based on photoplethysmography (PPG).

FIG. 1B shows an example of a blood pressure monitoring device based on photoacoustic plethysmography, which may be referred to herein as PAPG.

FIG. 2 is a block diagram that shows example components of an apparatus according to some disclosed implementations.

FIG. 3 is a flow diagram that shows examples of some disclosed operations.

FIG. 4A shows an example of a range-gate window (RGW) selected to receive acoustic waves emitted from a range of different depths.

FIG. 4B shows examples of multiple acquisition time delays being selected to receive acoustic waves emitted from different depths.

FIGS. 5A, 5B and 5C show examples of devices configured to receive acoustic waves emitted from different depths.

FIG. 6 shows examples of heart rate waveform (HRW) features that may be extracted according to some implementations.

FIG. 7 shows examples of devices that may be used in a system for estimating blood pressure based, at least in part, on pulse transit time (PTT).

FIG. 8 shows a cross-sectional side view of a diagrammatic representation of a portion of an artery 800 through which a pulse 802 is propagating.

FIG. 9A shows an example ambulatory monitoring device designed to be worn around a wrist according to some implementations.

FIG. 9B shows an example ambulatory monitoring device 900 designed to be worn around a finger according to some implementations.

FIG. 9C shows an example ambulatory monitoring device 900 designed to reside on an earbud according to some implementations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

In recent years, a variety of different devices for biometric and biomedical applications, including health and wellness monitoring, biometric authentication, etc., have appeared on the marketplace. Some such devices use high-intensity light, such as laser light. For example, some health monitoring systems and some biometric authentication systems may use high-intensity light for liveness detection, such as for cardiac pulse detection. Some devices may be configured to use high-intensity light for blood oxygen estimation, heart rate monitoring, blood pressure monitoring, etc. Some such devices may use high-intensity light for blood pressure monitoring based on photoplethysmography (PPG) or photoacoustic plethysmography (PAPG).

Such non-invasive devices have various advantages over previously-deployed devices such as cuff-based or catheter-based blood pressure measurement devices. However, many of these devices are sold to the general public, not to medical professionals. As such, there is an increased likelihood of misuse, or careless use, which could potentially cause high-intensity light, such as laser light, to be directed towards a person's eye or towards another vulnerable area (such as the eye of a pet). This could cause discomfort or, in some cases, eye injury.

Some disclosed devices include a light source system, a target detection system; and a control system. The control system may be configured to receive target detection data from the target detection system and to estimate the presence or absence of a biological target based, at least in part, on the target detection data. The control system may be configured to enable or disable the light source system based, at least in part, on the estimated presence or absence of the biological target. For example, if the biological target is detected, the control system may enable the light source system.

Particular implementations of the subject matter described in this disclosure can be implemented to realize one or more of the following potential advantages. Some implementations are configured to mitigate the risk of eye injury that could otherwise be caused by devices that use high-intensity light, such as laser light. Some such implementations also may save power by only enabling the light source system when a biological target is in position.

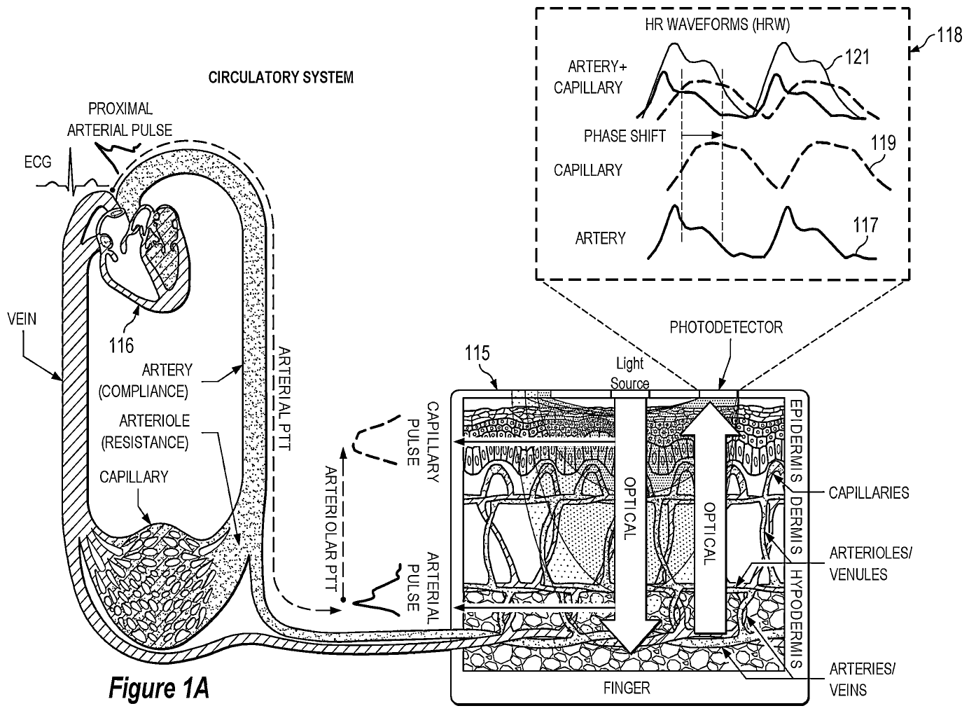

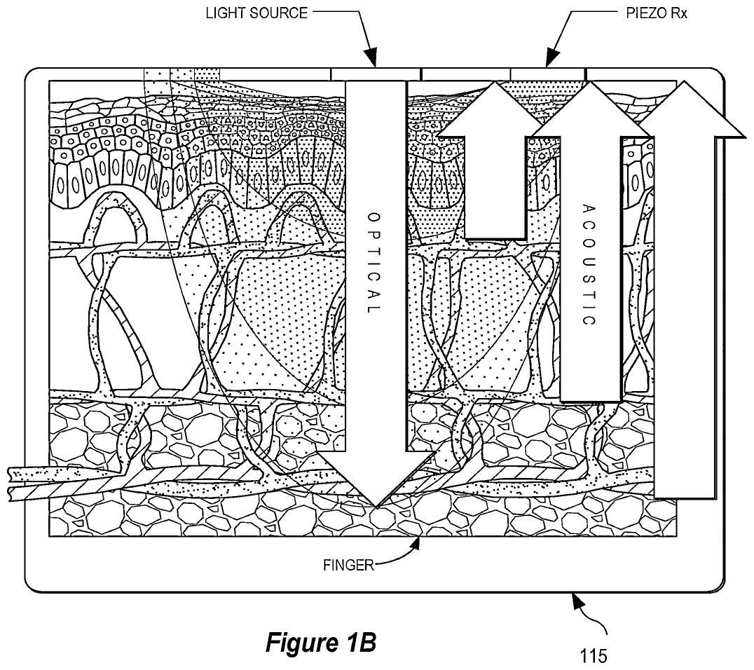

FIG. 1A shows an example of a blood pressure monitoring device based on photoplethysmography (PPG). FIG. 1A shows examples of arteries, veins, arterioles, venules and capillaries of a circulatory system, including those inside a finger 115. In the example shown in FIG. 1A, an electrocardiogram (ECG) sensor has detected a proximal arterial pulse near the heart 116. Some examples are described below of measurement of the arterial pulse transit time (PTT) according to arterial pulses measured by two sensors, one of which may be an electrocardiogram sensor in some implementations.

According to the example shown in FIG. 1A, a light source that includes one or more lasers or light-emitting diodes (LEDs) has transmitted light (in some examples, green, red, and/or near-infrared (NIR) light) that has penetrated the tissues of the finger 115 in an illuminated zone. Reflections from these tissues, detected by the photodetector, may be used to detect volumetric changes in the blood of the illuminated zone of the finger 115 that correspond to heart rate waveforms.

As shown in the heart rate waveform graphs 118 of FIG. 1A, the capillary heart rate waveform 119 is differently-shaped and phase-shifted relative to the artery heart rate waveform 117. In this simple example, the detected heart rate waveform 121 is a combination of the capillary heart rate waveform 119 and the artery heart rate waveform 117. In some instances, the responses of one or more other blood vessels may also be part of the heart rate waveform 121 detected by a PPG-based blood pressure monitoring device. PPG-based blood pressure monitoring devices are not optimal because PPG superimposes data corresponding to the blood volume of all illuminated blood vessels, each of which exhibit different and time-shifted blood volume changes.

Nonetheless, there are many deployed PPG-based blood pressure monitoring devices. Many such devices are configured to emit high-intensity light, such as laser light. Accordingly, such devices could advantageously be modified to implement the methods disclosed herein, in to mitigate the risk of eye injury, etc., that could otherwise be caused by such devices.

FIG. 1B shows an example of a blood pressure monitoring device based on photoacoustic plethysmography, which may be referred to herein as PAPG. FIG. 1B shows the same examples of arteries, veins, arterioles, venules and capillaries inside the finger 115 that are shown in FIG. 1A. In some examples, the light source shown in FIG. 1B may be, or may include, one or more LEDs, one or more laser diodes, etc. In this example, as in FIG. 1A, the light source has transmitted light (in some examples, green, red, and/or near-infrared (NIR) light) that has penetrated the tissues of the finger 115 in an illuminated zone.

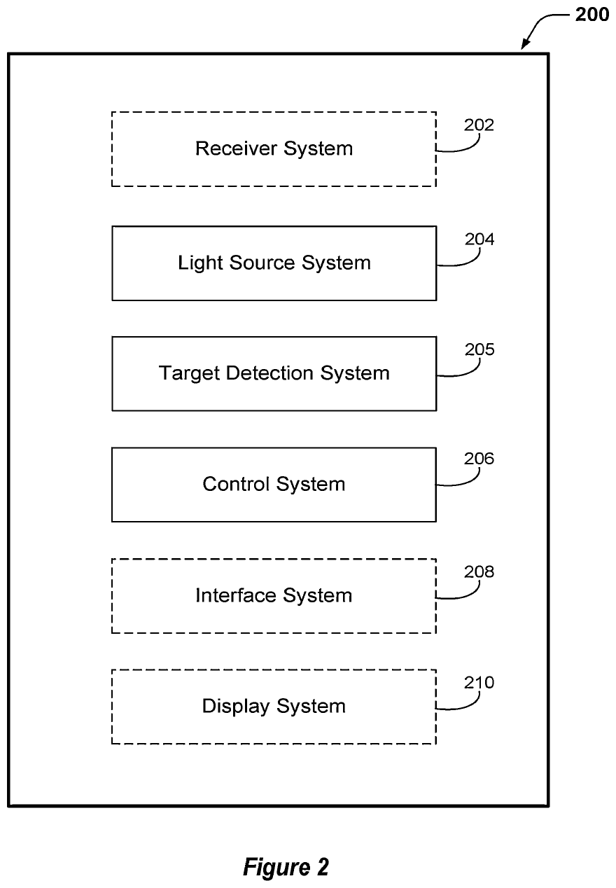

In the example shown in FIG. 1B, blood vessels (and components of the blood itself) are heated by the incident light from the light source and are emitting acoustic waves. In this example, the emitted acoustic waves include ultrasonic waves. According to this implementation, the acoustic wave emissions are being detected by an ultrasonic receiver, which is a piezoelectric receiver in this example. Photoacoustic emissions from the illuminated tissues, detected by the piezoelectric receiver, may be used to detect volumetric changes in the blood of the illuminated zone of the finger 115 that correspond to heart rate waveforms. Although some of the tissue areas shown to be illuminated are offset from those shown to be producing photoacoustic emissions, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination. In some examples, the ultrasonic receiver may be an instance of the receiver system 202 that is described below with reference to FIG. 2.

One important difference between the PPG-based system of FIG. 1A and the PAPG-based method of FIG. 1B is that the acoustic waves shown in FIG. 1B travel much more slowly than the reflected light waves shown in FIG. 1A. Accordingly, depth discrimination based on the arrival times of the acoustic waves shown in FIG. 1B is possible, whereas depth discrimination based on the arrival times of the light waves shown in FIG. 1A may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

PAPG-based blood pressure monitoring devices may be configured to emit high-intensity light, such as laser light. Accordingly, such devices could advantageously be modified to implement the methods disclosed herein, in to mitigate the risk of eye injury, etc., that could otherwise be caused by such devices.

FIG. 2 is a block diagram that shows example components of an apparatus according to some disclosed implementations. In this example, the apparatus 200 includes a light source system 204, a target detection system 205 and a control system 206. Although not shown in FIG. 2, the apparatus 200 may include a substrate. Some implementations of the apparatus 200 may include a receiver system 202, an interface system 208 and/or a display system 210.

Various examples of receiver systems 202 are disclosed herein, some of which may include ultrasonic receiver systems, optical receiver systems, or combinations thereof. In some implementations that include an ultrasonic receiver system, the ultrasonic receiver and an ultrasonic transmitter may be combined in an ultrasonic transceiver. In some examples, the receiver system 202 may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 202 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 202 may be, or may include, an ultrasonic receiver array. In some examples, the apparatus 200 may include one or more separate ultrasonic transmitter elements. In some such examples, the ultrasonic transmitter(s) may include an ultrasonic plane-wave generator.

The light source system 204 may, in some examples, include an array of light-emitting diodes. In some implementations, the light source system 204 may include one or more laser diodes. According to some implementations, the light source system 204 may include one or more vertical-cavity surface-emitting lasers (VCSELs). In some implementations, the light source system may include one or more neodymium-doped yttrium aluminum garnet (Nd:YAG) lasers. In some examples, the light source system 204 may be configured for emitting a plurality of light pulses at a pulse repetition frequency between 10 Hz and 100 kHz.

According to some implementations, the light source system may include at least one infrared, red, green, blue, white or ultraviolet light-emitting diode. In some implementations, the light source system 204 may include one or more laser diodes. For example, the light source system 204 may include at least one infrared, red, green, blue, white or ultraviolet laser diode.

In some implementations, the light source system 204 may be configured for emitting various wavelengths of light, which may be selectable to trigger acoustic wave emissions primarily from a particular type of material. For example, because the hemoglobin in blood absorbs near-infrared light very strongly, in some implementations the light source system 204 may be configured for emitting one or more wavelengths of light in the near-infrared range, in order to trigger acoustic wave emissions from hemoglobin. However, in some examples the control system 206 may control the wavelength(s) of light emitted by the light source system 204 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system 202. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying RGDs) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photo acoustically.

According to some implementations, the light source system 204 may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system 204 may be configured for emitting a plurality of light pulses at a pulse repetition frequency between about 1 MHz and about 100 MHz. Alternatively, or additionally, in some implementations the light source system 204 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system 204 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 204. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

In this example, the apparatus 200 includes a target detection system 205. The target detection system 205 may be configured to provide target detection data to the control system 206. The control system 206 may be configured to detect, or to estimate, the presence of a biological target, such as a finger, a wrist or an ear, based at least in part on the target detection data.

The specific component or components of the target detection system 205 may vary according to the particular implementation. According to some examples, the target detection system 205 may be, or may include, a touch sensor system. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the target detection system 205 may be, or may include, a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some such examples, the force sensor system may be integrated into circuitry of the ultrasonic fingerprint sensor.

According to some examples, the target detection system 205 may be, or may include, one or more mechanical switches, one or more electrical switches, one or more magnetic switches, one or more magnets configured for electrical continuity, or combinations thereof.

In some examples, the target detection system 205 may be, or may include, an optical sensor system, one or more cameras, or a combination thereof. According to some examples in which the target detection system 205 includes an optical sensor system, the control system 206 may be configured to control an intensity of light emitted by the light source system 204 based, at least in part, on optical sensor data from the optical sensor system.

According to some examples, the target detection system 205 may be, or may include, a liveness detection system. In some such examples, the target detection system 205 may include a cardiac pulse detection system. According to some examples, the cardiac pulse detection system may include a camera system, an ultrasonic pulse detection system, an optical pulse detection system, a photoacoustic pulse detection system, a photoplethysmography system, a microphone system, a ballistocardiogram sensor system, or combinations thereof. In some such examples, such as the ultrasonic pulse detection system, optical pulse detection system, photoacoustic pulse detection system, and photoplethysmography system examples, the target detection system 205 may include at least a portion of the receiver system 202, the light source system 204, or combinations thereof, despite the fact that the receiver system 202, the light source system 204 and the target detection system 205 are shown as separate blocks in FIG. 2.

In some examples, the target detection system 205 may be configured to produce a first instance of target detection data at a first time and to produce a second instance of target detection data at a second time. The time interval between the first time and the second time may be referred to herein as a target detection latency period. In some examples, the target detection latency period may be less than a pulse repetition frequency of the light source system 204.

The control system 206 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 206 also may include (and/or be configured for communication with) one or more memory devices, such as one or more random access memory (RAM) devices, read-only memory (ROM) devices, etc. Accordingly, the apparatus 200 may have a memory system that includes one or more memory devices, though the memory system is not shown in FIG. 2. The control system 206 may be configured for receiving and processing data from the receiver system 202, e.g., as described below. If the apparatus 200 includes an ultrasonic transmitter, the control system 206 may be configured for controlling the ultrasonic transmitter. In some implementations, functionality of the control system 206 may be partitioned between one or more controllers or processors, such as a dedicated sensor controller and an applications processor of a mobile device.

As noted elsewhere herein, the control system 206 may be configured to control the light source system 204 based, at least in part, on target detection data from the target detection system 205. In some such examples, the control system 206 may be configured to estimate the presence or absence of a biological target based, at least in part, on the target detection data. In some such examples, the control system 206 may be configured to enable or disable the light source system based, at least in part, on an estimation of the presence or absence of the biological target.

In some implementations, the control system may be configured for selecting one or more wavelengths of light for the plurality of light pulses. For example, because the hemoglobin in blood absorbs near-infrared light very strongly, in some implementations the control system may be configured for selecting one or more wavelengths of light in the near-infrared range, in order to trigger acoustic wave emissions from hemoglobin. According to some examples, the control system may be configured for selecting a light intensity associated with one or more selected wavelengths. For example, the control system may be configured for selecting one or more wavelengths of light and light intensities associated with each selected wavelength to generate acoustic wave emissions from one or more portions of the target object. In some examples, the control system may be configured for selecting the one or more wavelengths of light to evaluate one or more characteristics of the target object, e.g., to evaluate blood oxygen levels.

Some implementations of the apparatus 200 may include the interface system 208. In some examples, the interface system 208 may include a wireless interface system. In some implementations, the interface system 208 may include a user interface system, one or more network interfaces, one or more interfaces between the control system 206 and a memory system and/or one or more interfaces between the control system 206 and one or more external device interfaces (e.g., ports or applications processors). According to some examples in which the interface system 208 is present and includes a user interface system, the user interface system may include a microphone system, a haptic feedback system, a voice command system, one or more displays, or combinations thereof.

According to some examples, the apparatus 200 may include a display system 210 that includes one or more displays. For example, the display system 210 may include one or more LED displays, such as one or more organic LED (OLED) displays.

The apparatus 200 may be used in a variety of different contexts, many examples of which are disclosed herein. For example, in some implementations a mobile device may include the apparatus 200. In some implementations, a wearable device may include the apparatus 200. The wearable device may, for example, be a bracelet, an armband, a wristband, a watch, a ring, a headband or a patch.

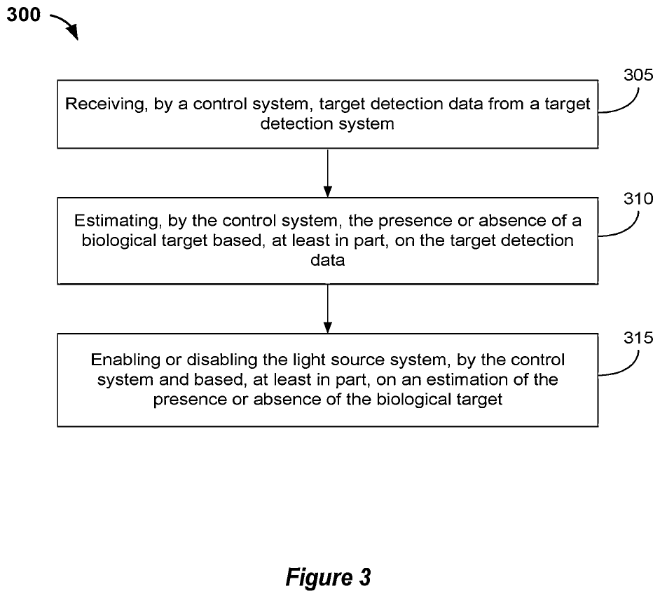

FIG. 3 is a flow diagram that shows examples of some disclosed operations. The blocks of FIG. 3 (and those of other flow diagrams provided herein) may, for example, be performed by the apparatus 200 of FIG. 2 or by a similar apparatus. As with other methods disclosed herein, the method outlined in FIG. 3 may include more or fewer blocks than indicated. Moreover, the blocks of methods disclosed herein are not necessarily performed in the order indicated. In some instances, one or more of the blocks shown in FIG. 3 may be performed concurrently.

In this example, block 305 involves receiving, by a control system, target detection data from a target detection system. According to some examples, the target detection system may be an instance of the target detection system 205 and the control system may be an instance of the control system 206. In some examples, block 305 may involve receiving touch sensor data from a touch sensor system. According to some examples, block 305 may involve receiving force sensor data from a force sensor system. In some examples, block 305 may involve receiving data from, or data corresponding to, one or more mechanical switches, one or more electrical switches, one or more magnetic switches, one or more magnets configured for electrical continuity, an optical sensor system, one or more cameras, or combinations thereof.

In some examples, block 305 may involve receiving liveness data from a liveness detection system. According to some examples, block 305 may involve receiving cardiac pulse data from a cardiac pulse detection system.

According to this implementation, block 310 involves estimating, by the control system, the presence or absence of a biological target based, at least in part, on the target detection data. The estimation of block 310 may vary according to the particular implementation. The estimation of block 310 may vary according to the particular type or types of target detection data received from the target detection system. If the target detection data includes only force sensor data, the estimation of block 310 may, in some examples, involve assuming that the force sensor data corresponds to a biological target pressing on the apparatus 200 in the area of a force sensor. If the target detection data includes only data from, or corresponding to, one or more mechanical switches, one or more electrical switches, one or more magnetic switches, one or more magnets configured for electrical continuity, etc., the estimation of block 310 may, in some examples, involve assuming that a wrist band, a finger band, etc., has or has not been attached to a biological target, depending on whether the switch is closed or open.

If the target detection data includes only touch sensor data, the estimation of block 310 may, in some examples, involve assuming that the touch sensor data corresponds to a biological target touching the apparatus 200 in the area of a touch sensor. However, if the target detection data includes touch sensor data from a touch sensor array, the estimation of block 310 may involve determining an area corresponding to the touch sensor data. In some examples, block 310 may involve estimating whether the area corresponds to the shape and size of a finger or other digit.

If the target detection data includes optical sensor data, camera data, etc., the estimation of block 310 may, in some examples, involve estimating whether image data from an optical sensor, a camera data, etc., corresponds to a finger or other digit, a wrist, a human ear, or other biological target. If the target detection data includes liveness data from a liveness detection system, the estimation of block 310 may, in some examples, involve estimating whether an object proximate, or on, the apparatus in the vicinity of the liveness detection system is part of a living being.

According to some examples, the estimation of block 310 may involve estimating a position of a biological target with reference to at least a portion of the light source system. For example, if a portion of the light source system is configured to emit high-intensity light, such as laser pulses, block 310 may involve an estimation of whether the biological target is placed on a portion of the apparatus 200 that corresponds with the portion of the light source system that is configured to emit high-intensity light. In some such examples, block 310 may involve an estimation of whether the biological target is covering an area that corresponds with the portion of the light source system that is configured to emit high-intensity light, such that the high-intensity light is unlikely to be directed to a human eye or another vulnerable area.

In this example, block 315 involves enabling or disabling the light source system, by the control system and based, at least in part, on an estimation of the presence or absence of the biological target. According to some examples, if it is estimated in block 310 that a biological object is proximate a portion of the apparatus 200 that corresponds with at least a portion of the light source system, block 315 may involve enabling the light source system. According to some examples, if it is estimated in block 310 that a biological object is covering a portion of the apparatus 200 that corresponds with a portion of the light source system that is configured to emit high-intensity light, block 315 may involve enabling the light source system. In some examples, if it is estimated in block 310 that a biological object is not proximate a portion of the apparatus 200 that corresponds with at least a portion of the light source system, block 315 may involve disabling the light source system.

According to some examples, method 300 may involve controlling (for example, by the control system) the light source system to emit one or more light pulses towards the biological target. In some such examples, method 300 may involve performing one or more types of bio-sensing functionality, biometric functionality, or combinations thereof, based on one or more responses of the biological target to the one or more light pulses. In some examples, method 300 may involve receiving, by the control system, ultrasonic receiver signals from an ultrasonic receiver system corresponding to ultrasound caused by the one or more responses of the biological target to the one or more light pulses. According to some examples, method 300 may involve providing, by the control system, photoacoustic imaging functionality, photoacoustic-based blood pressure estimation functionality, a photoacoustic-based authentication process or combinations thereof, based at least in part on the ultrasonic receiver signals.

According to some examples, the control system may be configured for discriminating between vein heart rate waveforms and artery heart rate waveforms by obtaining depth-discriminated signals. According to some such examples, receiving the signals from the piezoelectric receiver involves obtaining depth-discriminated signals by applying first through N^{th} acquisition time delays and receiving first through N^{th} signals during first through N^{th} acquisition time windows, each of the first through N^{th} acquisition time windows occurring after a corresponding one of the first through N^{th} acquisition time delays, wherein N is an integer greater than one. The control system may be configured for determining the first subset of detected heart rate waveforms and the second subset of detected heart rate waveforms based, at least in part, on the depth-discriminated signals.

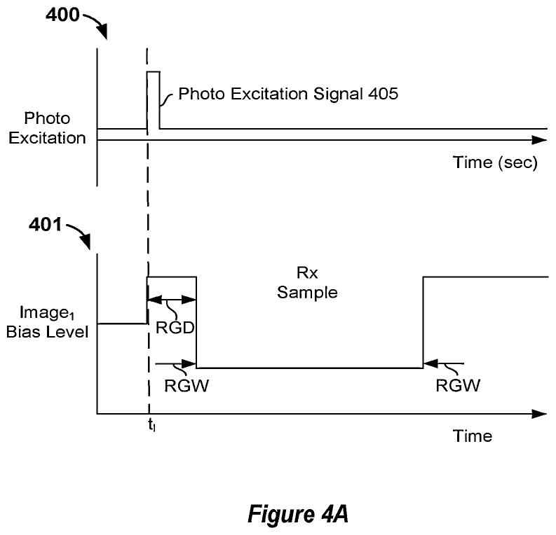

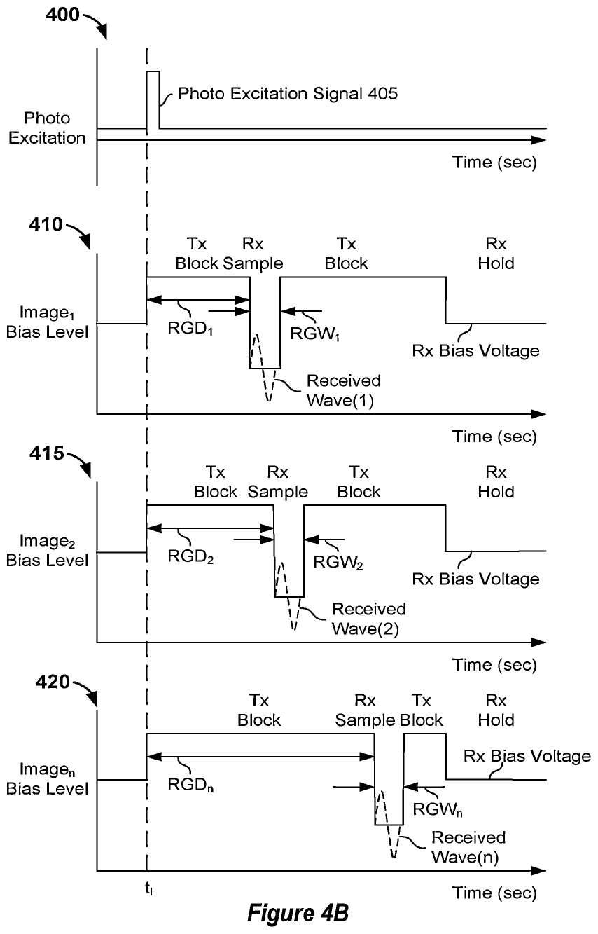

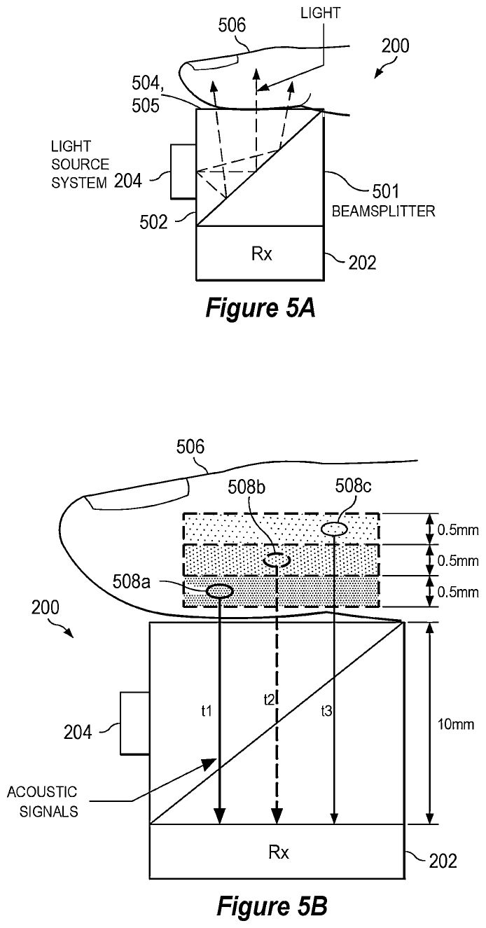

According to some examples, the control system may be configured for discriminating between vein heart rate waveforms and artery heart rate waveforms by obtaining depth-discriminated signals. FIG. 4A shows an example of a range-gate window (RGW) selected to receive acoustic waves emitted from a range of different depths. The acquisition time delay or range gate delay (which is labeled “RGD” in FIG. 4B) is measured from the beginning time t₁ of the photo-excitation signal 405 shown in graph 400. The RGD may, for example, be selected to correspond with the time required for photoacoustic emissions from a shallowest target of interest to reach a receiver, e.g., as described below with reference to FIGS. 5A and 5B. Accordingly, the RGD may depend on the particular arrangement of the apparatus being used to receive the photoacoustic emissions, including the thickness of the layer(s) between the target object and the receiver and the speed of sound of the layer(s) between the target object and the receiver. The graph 401 depicts a time after RGD during which emitted acoustic waves may be received and sampled by an ultrasonic receiver during an acquisition time window (also known as a range-gate window or a range-gate width) of RGW. In some implementations, the RGW may be 10 microseconds. Other implementations may have larger or smaller RGWs.

In some examples, depth-discriminated signals may be obtained by a process of partitioning the acoustic waves received during the RGW into a plurality of smaller time windows. Each of the time windows may correspond to a depth range inside the target object from which the acoustic waves are received. In some examples, the depth range or thickness of each layer may be 0.5 mm. Assuming a speed of sound of 1.5 mm/microsecond, each 0.5 mm layer would correspond to a time slot of approximately 0.33 microseconds. However, the depth range may vary according to the particular implementation.

According to some alternative examples, receiving the signals from the piezoelectric receiver involves obtaining depth-discriminated signals by applying first through N^{th} acquisition time delays and receiving first through N^{th} signals during first through N^{th} acquisition time windows, each of the first through N^{th} acquisition time windows occurring after a corresponding one of the first through N^{th} acquisition time delays, wherein N is an integer greater than one. The control system may be configured for determining the first subset of detected heart rate waveforms and the second subset of detected heart rate waveforms based, at least in part, on the depth-discriminated signals.

FIG. 4B shows examples of multiple acquisition time delays being selected to receive acoustic waves emitted from different depths. In these examples, each of the acquisition time delays (which are labeled range-gate delays or RGDs in FIG. 4B) is measured from the beginning time t₁ of the photo-excitation signal 405 shown in graph 400. The graph 410 depicts emitted acoustic waves (received wave (1) is one example) that may be received by an ultrasonic sensor array at an acquisition time delay RGD₁ and sampled during an acquisition time window (also known as a range-gate window or a range-gate width) of RGW₁. Such acoustic waves will generally be emitted from a relatively shallower portion of a target object proximate, or positioned upon, a platen of the biometric system.

Graph 415 depicts emitted acoustic waves (received wave (2) is one example) that are received by the ultrasonic sensor array at an acquisition time delay RGD₂ (with RGD₂>RGD₁) and sampled during an acquisition time window of RGW₂. Such acoustic waves will generally be emitted from a relatively deeper portion of the target object.

Graph 420 depicts emitted acoustic waves (received wave (n) is one example) that are received at an acquisition time delay RGDₙ (with RGDₙ>RGD₂>RGD₁) and sampled during an acquisition time window of RGWₙ. Such acoustic waves will generally be emitted from a still deeper portion of the target object. Range-gate delays are typically integer multiples of a clock period. A clock frequency of 128 MHz, for example, has a clock period of 7.8125 nanoseconds, and RGDs may range from under 10 nanoseconds to over 2000 nanoseconds. Similarly, the range-gate widths may also be integer multiples of the clock period, but are often much shorter than the RGD (e.g. less than about 50 nanoseconds) to capture returning signals while retaining good axial resolution. In some implementations, the acquisition time window (e.g. RGW) may be between 175 nanoseconds to 320 nanoseconds or more. In some examples, the RGW may be more or fewer nanoseconds, e.g., in the range of 25 nanoseconds to 1000 nanoseconds.

FIGS. 5A, 5B and 5C show examples of devices configured to receive acoustic waves emitted from different depths. The apparatus shown in FIGS. 5A-5C are examples of the apparatus 200 that is shown in FIG. 2. As with the other implementations shown and described herein, the types of elements, the arrangement of the elements and the dimensions of the elements illustrated in FIGS. 5A-5C are merely shown by way of example.

According to these examples, the apparatus 200 includes an ultrasonic receiver, which is an instance of the receiver system 202, a light source system 204 (which may include an LED in some examples), a target detection system 505 and a control system (which is not shown in FIGS. 5A-5C). In these examples, the target detection system 505 is an instance of the target detection system 205 that is described with reference to FIG. 2. According to the implementation shown in FIG. 5A, the apparatus 200 includes a beamsplitter 501 onto a side 502 to which the light source system 204 is mounted. In this instance, a finger 506 rests upon an adjacent surface 504 of the apparatus 200 and is detected by the target detection system 505. According to this example, the target detection system 505 provides target detection data to the control system indicating that the finger 506 is in contact with the surface 504. Based on the target detection data, the control system estimates that a biological target is present on the surface 504 and enables the light source system 204.

FIG. 5A shows light emitted from the light source system 204, part of which is reflected by the beamsplitter 501 and enters the finger 506. The range gate delay for this implementation and other implementations may, for example, be selected to correspond with the time required for photoacoustic emissions from a shallowest target of interest to reach a receiver. For example, in one configuration of the apparatus 200 which uses a 12.7 mm beamsplitter between the finger 506 and the ultrasonic receiver 202 (RX in FIG. 5A), the finger surface signal will arrive at the time it takes the acoustic waves to travel through the entire beamsplitter. Using the speed of sound of borosilicate glass of 5500 m/s as an approximate speed of sound for the beamsplitter and with the beamsplitter size of 12.7 mm, this time becomes 12.7 mm/5500 m/s or 2.3 us. Therefore, a range gate delay of 2.3 μs corresponds to the surface of the finger 506. To travel 1 mm into the finger 506, for example, using the speed of sound for tissue now of 1.5 mm/us, this time becomes 1 mm/1.5 mm/μs or ˜0.67 μs. Therefore, a range gate delay of ˜2.97 μs (2.3 μs+0.67 μs) would cause the ultrasonic receiver 202 to begin sampling acoustic waves reflected from a depth of approximately 1 mm below the outer surface of the finger 506.

FIG. 5B shows acoustic signals corresponding to photoacoustic emissions from tissues (e.g., blood and blood vessels) inside the finger 506, caused by the light that entered the finger 506. In the example shown in FIG. 5B, the acoustic signals originate from different depths (depths 508a, 508b and 508c) within the finger 506. Accordingly, the travel times t1, t2 and t3, from the depths 508a, 508b and 508c, respectively, to the ultrasonic receiver 202, are also different: in this instance, t3>t2>t1. Therefore, multiple acquisition time delays may be selected to receive acoustic waves emitted from the depths 508a, 508b and 508c, e.g., as shown in FIG. 4B and described above.

In the implementation shown in FIG. 5C, the positions of the light source system 204 and the receiver system 202 are reversed, as compared to the positions shown in FIG. 5A. According to this implementation, the apparatus 200 includes a translucent light pipe 510 through which light from the light source system 204 may pass. In this implementation, ultrasound that is generated within the finger 506 is reflected by an air/light pipe interface towards the receiver system 202. In this example, the finger 506 rests upon an adjacent surface 504 of the apparatus 200 and is detected by the target detection system 505. According to this example, the target detection system 505 provides target detection data to the control system indicating that the finger 506 is in contact with the surface 504. Based on the target detection data, the control system estimates that a biological target is present on the surface 504 and enables the light source system 204.

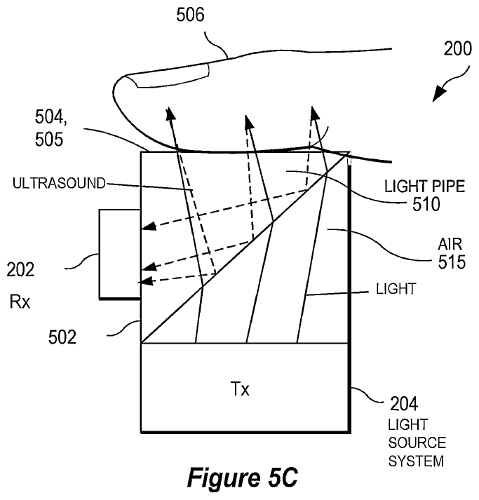

FIG. 6 shows examples of heart rate waveform (HRW) features that may be extracted according to some implementations. The horizontal axis of FIG. 6 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 6 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

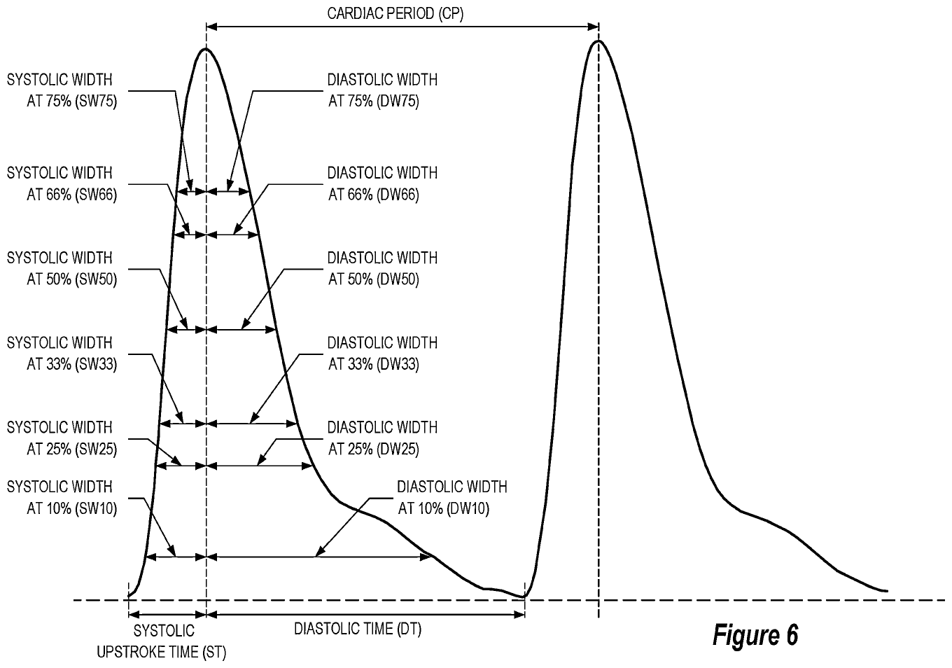

FIG. 7 shows examples of devices that may be used in a system for estimating blood pressure based, at least in part, on pulse transit time (PTT). As with other figures provided herein, the numbers, types and arrangements of elements are merely presented by way of example. According to this example, the system 700 includes at least two sensors. In this example, the system 700 includes at least an electrocardiogram sensor 705 and a device 710 that is configured to be mounted on a finger of the person 701. In this example, the device 710 is, or includes, an apparatus configured to perform at least some PAPG methods disclosed herein. For example, the device 710 may be, or may include, the apparatus 200 of FIG. 2 or a similar apparatus.

As noted in the graph 720, the PAT includes two components, the pre-ejection period (PEP, the time needed to convert the electrical signal into a mechanical pumping force and isovolumetric contraction to open the aortic valves) and the PTT. The starting time for the PAT can be estimated based on the QRS complex—an electrical signal characteristic of the electrical stimulation of the heart ventricles. As shown by the graph 720, in this example the beginning of a pulse arrival time (PAT) may be calculated according to an R-Wave peak measured by the electrocardiogram sensor 705 and the end of the PAT may be detected via analysis of signals provided by the device 710. In this example, the end of the PAT is assumed to correspond with an intersection between a tangent to a local minimum value detected by the device 710 and a tangent to a maximum slope/first derivative of the sensor signals after the time of the minimum value.

There are many known algorithms for blood pressure estimation based on the PTT and/or the PAT, some of which are summarized in Table 1 of Sharma, M., et al., Cuff-Less and Continuous Blood Pressure Monitoring: a Methodological Review (“Sharma”), in Multidisciplinary Digital Publishing Institute (MDPI) Technologies 2017, 5, 21, and which are described in the corresponding text on pages 5-10 of Sharma, both of which are hereby incorporated by reference.

Other implementations of the system 700 may not include the electrocardiogram sensor 705. In some such implementations, the device 715, which is configured to be mounted on a wrist of the person 701, may be, or may include, an apparatus configured to perform at least some PAPG methods disclosed herein. For example, the device 715 may be, or may include, the apparatus 200 of FIG. 2 or a similar apparatus. According to some such examples, the device 715 may include a light source system and two or more ultrasonic receivers. Examples are described below with reference to FIGS. 17A-17C. In some examples, the device 715 may include at least one array of ultrasonic receivers.

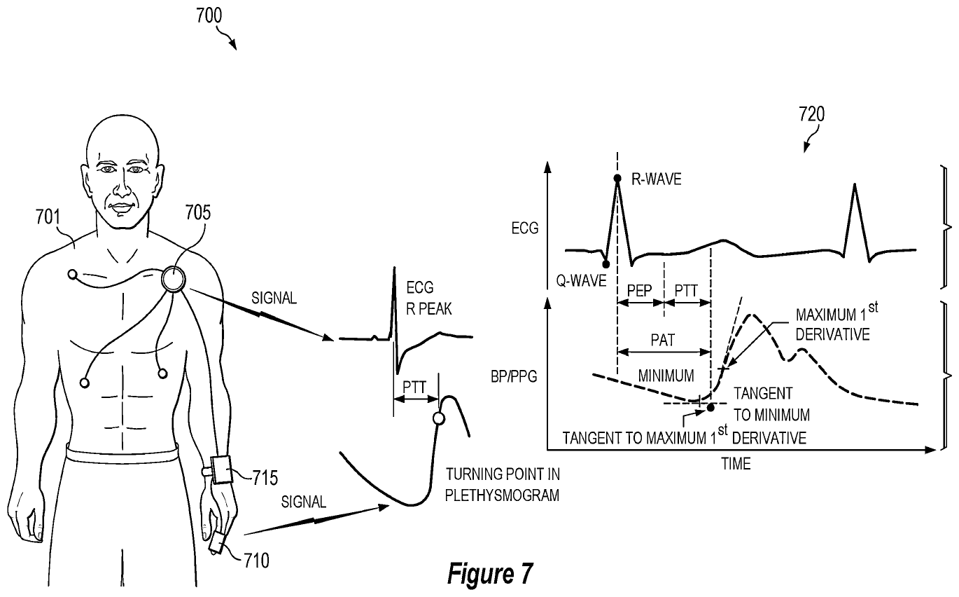

FIG. 8 shows a cross-sectional side view of a diagrammatic representation of a portion of an artery 800 through which a pulse 802 is propagating. The block arrow in FIG. 8 shows the direction of blood flow and pulse propagation. As diagrammatically shown, the propagating pulse 802 causes strain in the arterial walls 804, which is manifested in the form of an enlargement in the diameter (and consequently the cross-sectional area) of the arterial walls—referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length L of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. It will be appreciated that this PTT is different from the PTT that is described above with reference to FIG. 15. However, either version of the PTT may be used for the purpose of blood pressure estimation. Assuming that the physical distance ΔD between the first and the second physical locations is ascertainable, the PWV can be estimated as the quotient of the physical spatial distance ΔD traveled by the pulse divided by the time (PTT) the pulse takes in traversing the physical spatial distance ΔD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance ΔD through which the pulse propagates between the first physical location and the second physical location. More specifically, the PWV generally depends on a number of factors including the density of the blood ρ, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length ΔD between the two locations where the measurements are performed).

In traditional methods for obtaining PWV, the starting time of the pulse has been obtained at the heart using an electrocardiogram (ECG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 806 and a second arterial distension sensor 808 proximate first and second physical locations, respectively, along an artery of interest. In some particular implementations, the first arterial distension sensor 806 and the second arterial distension sensor 808 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance ΔD of separation between the first arterial distension sensor 806 and the second arterial distension sensor 808 (and consequently the distance between the first and the second locations along the artery) can be in the range of about 1 centimeter (cm) to tens of centimeters—long enough to distinguish the arrival of the pulse at the first physical location from the arrival of the pulse at the second physical location, but close enough to provide sufficient assurance of arterial consistency. In some specific implementations, the distance ΔD between the first and the second arterial distension sensors 806 and 808 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance ΔD between the first and the second arterial distension sensors 806 and 808 can be less than or equal to 1 cm, for example, about 0.1 cm, about 0.25 cm, about 0.5 cm or about 0.75 cm. By way of reference, a typical PWV can be about 15 meters per second (m/s). Using an ambulatory monitoring device in which the first and the second arterial distension sensors 806 and 808 are separated by a distance of about 5 cm, and assuming a PWV of about 15 m/s implies a PTT of approximately 3.3 milliseconds (ms).

The value of the magnitude of the distance ΔD between the first and the second arterial distension sensors 806 and 808, respectively, can be preprogrammed into a memory within a monitoring device that incorporates the sensors (for example, such as a memory of, or a memory configured for communication with, the control system 206 that is described above with reference to FIG. 2). As will be appreciated by a person of ordinary skill in the art, the spatial length L of a pulse can be greater than the distance ΔD from the first arterial distension sensor 806 to the second arterial distension sensor 808 in such implementations. As such, although the diagrammatic pulse 802 shown in FIG. 8 is shown as having a spatial length L comparable to the distance between the first arterial distension sensor 806 and the second arterial distension sensor 808, in actuality each pulse can typically have a spatial length L that is greater and even much greater than (for example, about an order of magnitude or more than) the distance ΔD between the first and the second arterial distension sensors 806 and 808.

### Sensing Architecture and Topology

In some implementations of the ambulatory monitoring devices disclosed herein, both the first arterial distension sensor 806 and the second arterial distension sensor 808 are sensors of the same sensor type. In some such implementations, the first arterial distension sensor 806 and the second arterial distension sensor 808 are identical sensors. In such implementations, each of the first arterial distension sensor 806 and the second arterial distension sensor 808 utilizes the same sensor technology with the same sensitivity to the arterial distension signal caused by the propagating pulses, and has the same time delays and sampling characteristics. In some implementations, each of the first arterial distension sensor 806 and the second arterial distension sensor 808 is configured for photoacoustic plethysmography (PAPG) sensing, e.g., as disclosed elsewhere herein. Some such implementations include a light source system and two or more ultrasonic receivers, which may be instances of the light source system 204 and the receiver system 202 of FIG. 2. In some implementations, each of the first arterial distension sensor 806 and the second arterial distension sensor 808 is configured for ultrasound sensing via the transmission of ultrasonic signals and the receipt of corresponding reflections. In some alternative implementations, each of the first arterial distension sensor 806 and the second arterial distension sensor 808 may be configured for impedance plethysmography (IPG) sensing, also referred to in biomedical contexts as bioimpedance sensing. In various implementations, whatever types of sensors are utilized, each of the first and the second arterial distension sensors 806 and 808 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude- and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb, a finger, etc., towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension. In some alternative implementations, light may be directed into a limb, a finger, etc., towards an artery, and ultrasound that is generated by biological tissue, responsive to the light, may be received by an ultrasonic receiver system.

In some implementations, regardless of the type of sensors utilized for the first arterial distension sensor 806 and the second arterial distension sensor 808, both the first arterial distension sensor 806 and the second arterial distension sensor 808 can be arranged, assembled or otherwise included within a single housing of a single ambulatory monitoring device. As described above, the housing and other components of the monitoring device can be configured such that when the monitoring device is affixed or otherwise physically coupled to a subject, both the first arterial distension sensor 806 and the second arterial distension sensor 808 are in contact with or in close proximity to the skin of the user at first and second locations, respectively, separated by a distance ΔD, and in some implementations, along a stretch of the artery between which various arterial properties can be assumed to be relatively constant. In various implementations, the housing of the ambulatory monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the ambulatory monitoring device facilitates and enables long-term wearing and monitoring (for example, over days, weeks or a month or more without interruption) of one or more biological characteristics of interest to obtain a better picture of such characteristics over extended durations of time, and generally, a better picture of the user's health.

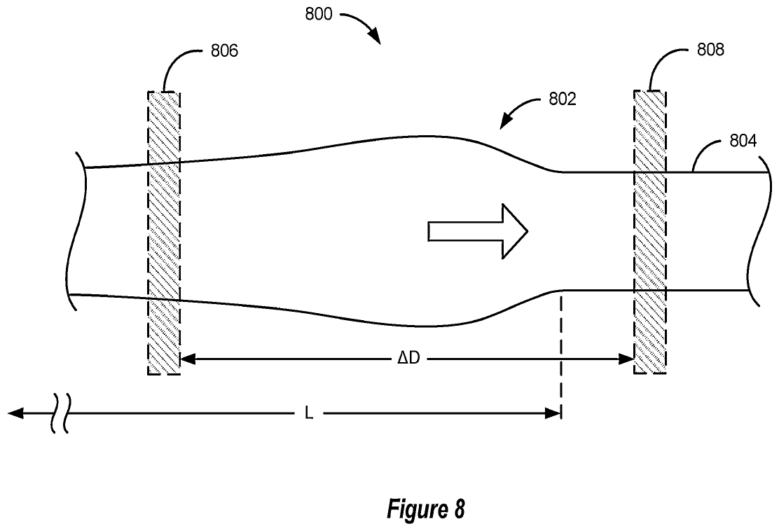

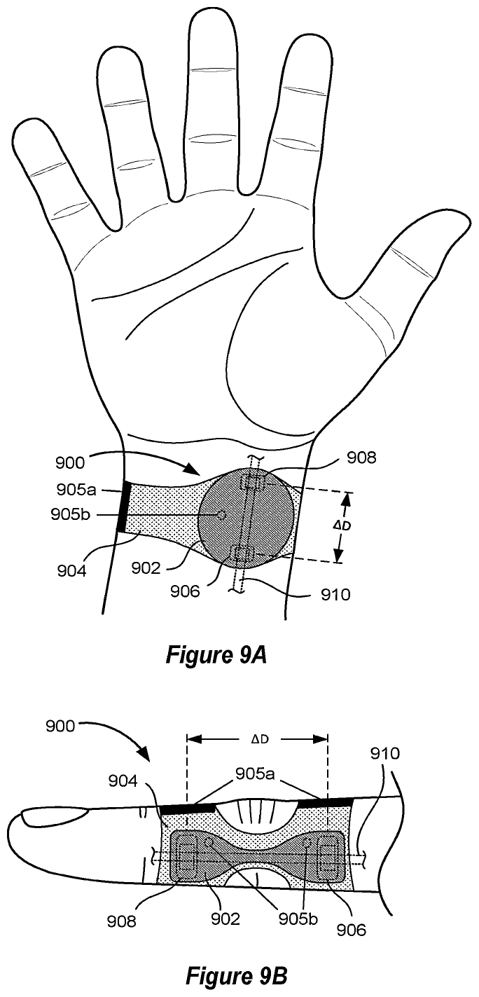

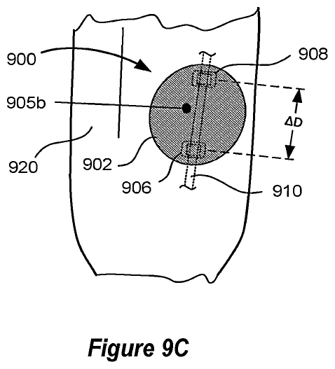

In some implementations, the ambulatory monitoring device can be positioned around a wrist of a user with a strap or band, similar to a watch or fitness/activity tracker. FIG. 9A shows an example ambulatory monitoring device 900 designed to be worn around a wrist according to some implementations. In this example, the ambulatory monitoring device 900 is an instance of the apparatus 200 of FIG. 2. In the illustrated example, the monitoring device 900 includes a housing 902 integrally formed with, coupled with or otherwise integrated with a band 904. The first and the second arterial distension sensors 906 and 908 may, in some instances, each include an instance of the receiver system 202 and a portion of the light source system 204 that are described above with reference to FIG. 2. In this example, the ambulatory monitoring device 900 is coupled around the wrist such that the first and the second arterial distension sensors 906 and 908 within the housing 902 are each positioned along a segment of the radial artery 910 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 906 and 908 are separated by a fixed distance ΔD. In some other implementations, the ambulatory monitoring device 900 can similarly be designed or adapted for positioning around a forearm, an upper arm, an ankle, a lower leg, an upper leg, or a finger (all of which are hereinafter referred to as “limbs”) using a strap or band.

According to this example, the ambulatory monitoring device 900 includes target detection system components 905a and 905b, each of which is an instance of the target detection system 205 of FIG. 2. In this example, the target detection system component 905a is configured to provide target detection data to a control system (not shown) indicating whether band 904 is fastened. In some examples, the target detection system component 905a may be, or may include, one or more mechanical switches, one or more electrical switches, one or more magnetic switches, one or more magnets configured for electrical continuity, or combinations thereof.

In this example, the target detection system component 905b is configured to provide target detection data to the control system indicating whether an object, such as a human wrist, is proximate the housing 902. In some examples, the target detection system component 905b may be, or may include, a touch sensor system, a force sensor system, an optical sensor system, one or more cameras, or combinations thereof. According to some examples, the first arterial distension sensor 906, the second arterial distension sensor 908, or both, may include a component of the target detection system 205. In some such examples, the first arterial distension sensor 906, the second arterial distension sensor 908, or both, may include a touch sensor system, a force sensor system, an optical sensor system, one or more cameras, or combinations thereof.

According to some examples in which the target detection system component 905b (or another component of the target detection system 205) includes an optical sensor system, the control system may be configured to control an intensity of light emitted by the light source system 204 based, at least in part, on optical sensor data from the optical sensor system. As noted above, in this example the first and the second arterial distension sensors 906 and 908 each include an instance of the receiver system 202 and a portion of the light source system 204 that are described above with reference to FIG. 2. Accordingly, in some examples in which the target detection system component 905b (or another component of the target detection system 205) includes an optical sensor system, the control system may be configured to control an intensity of light emitted by the first arterial distension sensor 906, the second arterial distension sensor 908, or both, based at least in part on optical sensor data from the optical sensor system.

FIG. 9B shows an example ambulatory monitoring device 900 designed to be worn around a finger according to some implementations. The first and the second arterial distension sensors 906 and 908 may, in some instances, each include an instance of the receiver system 202 and a portion of the light source system 204 that are described above with reference to FIG. 2.

According to this example, the ambulatory monitoring device 900 includes target detection system components 905a and 905b, each of which is an instance of the target detection system 205 of FIG. 2. In this example, the target detection system component 905a is configured to provide target detection data to a control system (not shown) indicating whether band 904 is fastened. In some examples, the target detection system component 905a may be, or may include, one or more mechanical switches, one or more electrical switches, one or more magnetic switches, one or more magnets configured for electrical continuity, or combinations thereof.

In this example, the target detection system components 905b are configured to provide target detection data to the control system indicating whether an object, such as a finger or other digit, is proximate the housing 902. In some examples, the target detection system components 905b may be, or may include, a touch sensor system, a force sensor system, an optical sensor system, one or more cameras, or combinations thereof. According to some examples, the first arterial distension sensor 906, the second arterial distension sensor 908, or both, may include a component of the target detection system 205. In some such examples, the first arterial distension sensor 906, the second arterial distension sensor 908, or both, may include a touch sensor system, a force sensor system, an optical sensor system, one or more cameras, or combinations thereof.

According to some examples in which one or more of the target detection system components 905b (or another component of the target detection system 205) includes an optical sensor system, the control system may be configured to control an intensity of light emitted by the light source system 204 based, at least in part, on optical sensor data from the optical sensor system. As noted above, in this example the first and the second arterial distension sensors 906 and 908 each include an instance of the receiver system 202 and a portion of the light source system 204 that are described above with reference to FIG. 2. Accordingly, in some examples in which one or more of the target detection system components 905b (or another component of the target detection system 205) includes an optical sensor system, the control system may be configured to control an intensity of light emitted by the first arterial distension sensor 906, the second arterial distension sensor 908, or both, based at least in part on optical sensor data from the optical sensor system.

In some other implementations, the ambulatory monitoring devices disclosed herein can be positioned on a region of interest of the user without the use of a strap or band. For example, the first and the second arterial distension sensors 906 and 908 and other components of the monitoring device can be enclosed in a housing that is secured to the skin of a region of interest of the user using an adhesive or other suitable attachment mechanism (an example of a “patch” monitoring device).

FIG. 9C shows an example ambulatory monitoring device 900 designed to reside on an earbud according to some implementations. According to this example, the ambulatory monitoring device 900 is coupled to the housing of an earbud 920. The first and second arterial distension sensors 906 and 908 may, in some instances, each include an instance of the receiver system 202 and a portion of the light source system 204 that are described above with reference to FIG. 2.

According to this example, the ambulatory monitoring device 900 includes target detection system component 905b, which is an instance of the target detection system 205 of FIG. 2. In this example, the target detection system component 905b is configured to provide target detection data to the control system indicating whether an object, such as a human ear, is proximate the housing 902. In some examples, the target detection system component 905b may be, or may include, a touch sensor system, a force sensor system, an optical sensor system, one or more cameras, or combinations thereof.

Implementation examples are described in the following numbered clauses:

1. An apparatus, including: a light source system; a target detection system; and a control system configured to communicate with the light source system and the target detection system, the control system being further configured to: receive target detection data from the target detection system; estimate a presence or absence of a biological target based, at least in part, on the target detection data; and enable or disable the light source system based, at least in part, on the estimation of the presence or absence of the biological target.

2. The apparatus of clause 1, where the light source system includes one or more lasers or laser diodes.

3. The apparatus of clause 1 or clause 2, where the light source system includes one or more light-emitting diodes.

4. The apparatus of any one of clauses 1-3, where the control system is further configured to control the light source system to emit one or more light pulses towards the biological target and to perform one or more types of bio-sensing functionality, biometric functionality, or combinations thereof, based on one or more responses of the biological target to the one or more light pulses.

5. The apparatus of clause 4, further including an ultrasonic receiver system, where the control system is further configured to receive ultrasonic receiver signals from the ultrasonic receiver system corresponding to ultrasound caused by the one or more responses of the biological target to the one or more light pulses.

6. The apparatus of clause 5, where the control system is further configured to provide photoacoustic imaging functionality, photoacoustic-based blood pressure estimation functionality, a photoacoustic-based authentication process or combinations thereof, based at least in part on the ultrasonic receiver signals.

7. The apparatus of any one of clauses 1-6, where the target detection system includes a touch sensor system, a force sensor system, one or more mechanical switches, one or more electrical switches, one or more magnetic switches, one or more magnets configured for electrical continuity, an optical sensor system, one or more cameras, or combinations thereof.

8. The apparatus of clause 7, where the target detection system includes the optical sensor system and where the control system is further configured to control an intensity of light emitted by the light source system based, at least in part, on optical sensor data from the optical sensor system.

9. The apparatus of any one of clauses 1-8, where the target detection system includes a liveness detection system.

10. The apparatus of clause 9, where the liveness detection system includes a cardiac pulse detection system.

11. The apparatus of clause 10, where the cardiac pulse detection system includes a camera system, an ultrasonic pulse detection system, an optical pulse detection system, a photoacoustic pulse detection system, a photoplethysmography system, a microphone system, a ballistocardiogram sensor system, or combinations thereof.

12. The apparatus of any one of clauses 1-11, where the target detection system is configured to produce a first instance of target detection data at a first time and to produce a second instance of target detection data at a second time, and where a time interval between the first time and the second time is a target detection latency period.

13. The apparatus of clause 12, where the target detection latency period is less than a pulse repetition frequency of the light source system.

14. An apparatus, including: a light source system; a target detection system; and control means for: receiving target detection data from the target detection system; estimating a presence or absence of a biological target based, at least in part, on the target detection data; and enabling or disabling the light source system based, at least in part, on the estimation of the presence or absence of the biological target.

15. The apparatus of clause 14, where the light source system includes one or more lasers or laser diodes.

16. The apparatus of clause 14 or clause 15, where the light source system includes one or more light-emitting diodes.

17. The apparatus of any one of clauses 14-16, where the control means includes means for controlling the light source system to emit one or more light pulses towards the biological target and to perform one or more types of bio-sensing functionality, biometric functionality, or combinations thereof, based on one or more responses of the biological target to the one or more light pulses.

18. The apparatus of clause 17, further including an ultrasonic receiver system, where the control means includes means for receiving ultrasonic receiver signals from the ultrasonic receiver system corresponding to ultrasound caused by the one or more responses of the biological target to the one or more light pulses.

19. The apparatus of clause 18, where the control means includes means for providing photoacoustic imaging functionality, photoacoustic-based blood pressure estimation functionality, a photoacoustic-based authentication process or combinations thereof, based at least in part on the ultrasonic receiver signals.

20. The apparatus of any one of clauses 14-19, where the target detection system includes a touch sensor system, a force sensor system, one or more mechanical switches, one or more electrical switches, one or more magnetic switches, one or more magnets configured for electrical continuity, an optical sensor system, one or more cameras, or combinations thereof.

21. A method of controlling a light source system, including: receiving, by a control system, target detection data from a target detection system; estimating, by the control system, a presence or absence of a biological target based, at least in part, on the target detection data; and enabling or disabling the light source system, by the control system and based, at least in part, on the estimation of the presence or absence of the biological target.

22. The method of clause 21, further including controlling, by the control system, the light source system to emit one or more light pulses towards the biological target and performing one or more types of bio-sensing functionality, biometric functionality, or combinations thereof, based on one or more responses of the biological target to the one or more light pulses.

23. The method of clause 22, further including receiving, by the control system, ultrasonic receiver signals from an ultrasonic receiver system corresponding to ultrasound caused by the one or more responses of the biological target to the one or more light pulses.

24. The method of clause 23, further including providing, by the control system, photoacoustic imaging functionality, photoacoustic-based blood pressure estimation functionality, a photoacoustic-based authentication process or combinations thereof, based at least in part on the ultrasonic receiver signals.

25. The method of any one of clauses 21-24, further including producing, by the target detection system, a first instance of target detection data at a first time and a second instance of target detection data at a second time, and where a time interval between the first time and the second time is a target detection latency period.

26. The method of clause 25, where the target detection latency period is less than a pulse repetition frequency of the light source system.

27. One or more non-transitory media having instructions stored thereon for controlling one or more devices to perform a method of controlling a light source system, the method including: receiving, by a control system, target detection data from a target detection system; estimating, by the control system, a presence or absence of a biological target based, at least in part, on the target detection data; and enabling or disabling the light source system, by the control system and based, at least in part, on the estimation of the presence or absence of the biological target.

28. The one or more non-transitory media of clause 27, where the method further includes controlling, by the control system, the light source system to emit one or more light pulses towards the biological target and to perform one or more types of bio-sensing functionality, biometric functionality, or combinations thereof, based on one or more responses of the biological target to the one or more light pulses.

29. The one or more non-transitory media of clause 28, where the method further includes receiving, by the control system, ultrasonic receiver signals from an ultrasonic receiver system corresponding to ultrasound caused by the one or more responses of the biological target to the one or more light pulses.

30. The one or more non-transitory media of clause 29, where the method further includes providing, by the control system, photoacoustic imaging functionality, photoacoustic-based blood pressure estimation functionality, a photoacoustic-based authentication process or combinations thereof, based at least in part on the ultrasonic receiver signals.

31. The one or more non-transitory media of any one of clauses 27-30, where the method further includes producing, by the target detection system, a first instance of target detection data at a first time and a second instance of target detection data at a second time, and where a time interval between the first time and the second time is a target detection latency period.

32. The one or more non-transitory media of clause 31, where the target detection latency period is less than a pulse repetition frequency of the light source system.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

What is claimed is:

1. An apparatus, comprising:
a light source system;
a target detection system; and
a control system configured to communicate with the light source system and the target detection system, the control system being further configured to:
receive target detection data from the target detection system;
estimate a presence or absence of a biological target based, at least in part, on the target detection data; and
enable or disable the light source system based, at least in part, on the estimation of the presence or absence of the biological target.

2. The apparatus of claim 1, wherein the light source system includes one or more lasers or laser diodes.

3. The apparatus of claim 1, wherein the light source system includes one or more light-emitting diodes.

4. The apparatus of claim 1, wherein the control system is further configured to control the light source system to emit one or more light pulses towards the biological target and to perform one or more types of bio-sensing functionality, biometric functionality, or combinations thereof, based on one or more responses of the biological target to the one or more light pulses.

5. The apparatus of claim 4, further comprising an ultrasonic receiver system, wherein the control system is further configured to receive ultrasonic receiver signals from the ultrasonic receiver system corresponding to ultrasound caused by the one or more responses of the biological target to the one or more light pulses.

6. The apparatus of claim 5, wherein the control system is further configured to provide photoacoustic imaging functionality, photoacoustic-based blood pressure estimation functionality, a photoacoustic-based authentication process or combinations thereof, based at least in part on the ultrasonic receiver signals.

7. The apparatus of claim 1, wherein the target detection system includes a touch sensor system, a force sensor system, one or more mechanical switches, one or more electrical switches, one or more magnetic switches, one or more magnets configured for electrical continuity, an optical sensor system, one or more cameras, or combinations thereof.

8. The apparatus of claim 7, wherein the target detection system includes the optical sensor system and wherein the control system is further configured to control an intensity of light emitted by the light source system based, at least in part, on optical sensor data from the optical sensor system.

9. The apparatus of claim 1, wherein the target detection system includes a liveness detection system.

10. The apparatus of claim 9, wherein the liveness detection system includes a cardiac pulse detection system.

11. The apparatus of claim 10, wherein the cardiac pulse detection system includes a camera system, an ultrasonic pulse detection system, an optical pulse detection system, a photoacoustic pulse detection system, a photoplethysmography system, a microphone system, a ballistocardiogram sensor system, or combinations thereof.

12. The apparatus of claim 1, wherein the target detection system is configured to produce a first instance of target detection data at a first time and to produce a second instance of target detection data at a second time, and wherein a time interval between the first time and the second time is a target detection latency period.

13. The apparatus of claim 12, wherein the target detection latency period is less than a pulse repetition frequency of the light source system.

14. An apparatus, comprising:
a light source system;
a target detection system; and
control means for:
receiving target detection data from the target detection system;
estimating a presence or absence of a biological target based, at least in part, on the target detection data; and
enabling or disabling the light source system based, at least in part, on the estimation of the presence or absence of the biological target.

15. The apparatus of claim 14, wherein the light source system includes one or more lasers or laser diodes.

16. The apparatus of claim 14, wherein the light source system includes one or more light-emitting diodes.

17. The apparatus of claim 14, wherein the control means includes means for controlling the light source system to emit one or more light pulses towards the biological target and to perform one or more types of bio-sensing functionality, biometric functionality, or combinations thereof, based on one or more responses of the biological target to the one or more light pulses.

18. The apparatus of claim 17, further comprising an ultrasonic receiver system, wherein the control means includes means for receiving ultrasonic receiver signals from the ultrasonic receiver system corresponding to ultrasound caused by the one or more responses of the biological target to the one or more light pulses.

19. The apparatus of claim 18, wherein the control means includes means for providing photoacoustic imaging functionality, photoacoustic-based blood pressure estimation functionality, a photoacoustic-based authentication process or combinations thereof, based at least in part on the ultrasonic receiver signals.

20. The apparatus of claim 14, wherein the target detection system includes a touch sensor system, a force sensor system, one or more mechanical switches, one or more electrical switches, one or more magnetic switches, one or more magnets configured for electrical continuity, an optical sensor system, one or more cameras, or combinations thereof.

21. A method of controlling a light source system, comprising:
receiving, by a control system, target detection data from a target detection system;
estimating, by the control system, a presence or absence of a biological target based, at least in part, on the target detection data; and
enabling or disabling the light source system, by the control system and based, at least in part, on the estimation of the presence or absence of the biological target.

22. The method of claim 21, further comprising controlling, by the control system, the light source system to emit one or more light pulses towards the biological target and performing one or more types of bio-sensing functionality, biometric functionality, or combinations thereof, based on one or more responses of the biological target to the one or more light pulses.

23. The method of claim 22, further comprising receiving, by the control system, ultrasonic receiver signals from an ultrasonic receiver system corresponding to ultrasound caused by the one or more responses of the biological target to the one or more light pulses.

24. The method of claim 23, further comprising providing, by the control system, photoacoustic imaging functionality, photoacoustic-based blood pressure estimation functionality, a photoacoustic-based authentication process or combinations thereof, based at least in part on the ultrasonic receiver signals.

25. The method of claim 21, further comprising producing, by the target detection system, a first instance of target detection data at a first time and a second instance of target detection data at a second time, and wherein a time interval between the first time and the second time is a target detection latency period.

26. The method of claim 25, wherein the target detection latency period is less than a pulse repetition frequency of the light source system.

27. One or more non-transitory media having instructions stored thereon for controlling one or more devices to perform a method of controlling a light source system, the method comprising:
receiving, by a control system, target detection data from a target detection system;
estimating, by the control system, a presence or absence of a biological target based, at least in part, on the target detection data; and
enabling or disabling the light source system, by the control system and based, at least in part, on the estimation of the presence or absence of the biological target.

28. The one or more non-transitory media of claim 27, wherein the method further comprises controlling, by the control system, the light source system to emit one or more light pulses towards the biological target and to perform one or more types of bio-sensing functionality, biometric functionality, or combinations thereof, based on one or more responses of the biological target to the one or more light pulses.

29. The one or more non-transitory media of claim 28, wherein the method further comprises receiving, by the control system, ultrasonic receiver signals from an ultrasonic receiver system corresponding to ultrasound caused by the one or more responses of the biological target to the one or more light pulses.

30. The one or more non-transitory media of claim 29, wherein the method further comprises providing, by the control system, photoacoustic imaging functionality, photoacoustic-based blood pressure estimation functionality, a photoacoustic-based authentication process or combinations thereof, based at least in part on the ultrasonic receiver signals.

31. The one or more non-transitory media of claim 27, wherein the method further comprises producing, by the target detection system, a first instance of target detection data at a first time and a second instance of target detection data at a second time, and wherein a time interval between the first time and the second time is a target detection latency period.

32. The one or more non-transitory media of claim 31, wherein the target detection latency period is less than a pulse repetition frequency of the light source system.
